(19) United States
(12) Patent Application Publication
(10) Pub. No.: US 2023/0213507 A1
(43) Pub. Date: Jul. 6, 2023

(54) OPTICAL PROBE FOR BIO-SENSOR, OPTICAL BIO-SENSOR INCLUDING OPTICAL PROBE, AND METHOD FOR MANUFACTURING OPTICAL PROBE FOR BIO-SENSOR

(71) Applicant: Industry-Academic Cooperation Foundation of Ajou University, Suwon-si (KR)

(72) Inventors: Hyun-Chul YOON, Seoul (KR); Yong-Duk HAN, Seongnam-si (KR); Jae-Ho KIM, Suwon-si (KR)

(73) Assignee: Industry-Academic Cooperation Foundation of Ajou University, Suwon-si (KR)

(21) Appl. No.: 18/181,066

(22) Filed: Mar. 9, 2023

Related U.S. Application Data
(62) Division of application No. 16/065,147, filed on Jun. 22, 2018.

(30) Foreign Application Priority Data
Dec. 23, 2015 (KR) 10-2015-0184599

(51) Int. Cl. G01N 33/543; G01N 21/17

(52) U.S. Cl. CPC G01N 33/54373; G01N 21/1717

(57) ABSTRACT

An optical probe for a bio-sensor selectively conjugated to a target analyte and configured to retro-reflect incident light thereto is disclosed. The optical probe for the bio-sensor includes: a transparent core particle; a total-reflection inducing layer covering a portion of a surface of the core particle, the inducing layer is made of a material having a refractive index lower than a refractive index of the core; a modifying layer formed on the total-reflection inducing layer; and an analyte-sensing substance bound to the modifying layer, the sensing substance is selectively conjugated to the target analyte. This optical probe may serve as an excellent optical probe for both a non-spectral light source and a spectral light source.

### TECHNICAL FIELD

The present disclosure relates to an optical probe for a bio-sensor capable of optically sensing a presence or concentration of a target analyte, a bio-sensor including the optical probe, and a method for manufacturing the optical probe for a bio-sensor.

### RELATED ART

Although a bio-sensor and a Lab-on-A-Chip have emerged as a core feature for a disease diagnosis technology, biosensors other than a blood glucose sensor and a rapid kit for infectious diseases have not yet achieved a significant success in a biosensor market. Particularly, the optical bio-sensor developed so far uses an optical probe to detect a reaction and a binding at a bioreceptor capable of selectively reacting and binding with a target analyte to be detected. A typical optical probe includes an enzyme, a chromogenic dye, a metal nanoparticle, an organic fluorescent dye, an inorganic fluorescent nanoparticle, and the like. These optical probes provide, via colorings depending on the types thereof, spectroscopic optical signals indicating an intensity change of an absorption spectrum, a spectral shifting of the absorption spectrum, a fluorescence intensity change in a presence of an excitation light, and the like. Although, these spectroscopic optical signals contribute to very good signal sensitivity of the bio-sensor, it is necessary to use following components in order to detect these spectroscopic optical signals; for example, 1) a high-power short-wavelength laser light source or a light source combined a halogen lamp and a monochromator, 2) an excitation/emission filter adapted for a corresponding spectroscopic optical signal, 3) high-sensitivity light receiving elements such as a photomultiplier tube (PMT), and the like. These spectroscopic light source and optical components are expensive and require a high complexity and a high power. Therefore, it is difficult for the spectroscopic light source and optical components to be applied to a point-of-care-testing (POCT) optical bio-sensor operating under a resource-limited condition.

Thus, it is essential to develop a new optical signal detection, conversion, and analysis methodology using a non-spectroscopic analysis in order to embody an intuitive and commercialized POCT optical bio-sensor. The new optical analysis methodology must be able to induce an optical signal from a general light source as a non-spectroscopic source, such as white mixed light. The corresponding signal must be able to be converted and analyzed using an optical system with a minimum number of components including a low magnification optical microscope or a smartphone without any need for a separate spectroscopic filter of an expensive light receiving element.

### DISCLOSURE OF PRESENT DISCLOSURE

### Technical Purposes

One object of the present disclosure is to provide an optical probe which is able to generate a strong retro-reflective signal from an incident light and thus is applied to a non-spectroscopic bio-sensor.

Another object of the present disclosure is to provide a bio-sensor capable of detecting a presence or a concentration of a target analyte in a non-spectroscopic manner using the optical probe.

Still another object of the present disclosure is to provide a method for manufacturing the optical probe.

### Technical Solutions

In one aspect of the present disclosure, there is provided an optical probe for a bio-sensor selectively conjugated to a target analyte and configured to retro-reflect incident light thereto, wherein the optical probe for the bio-sensor including: a transparent core particle; a total-reflection inducing layer covering a portion of a surface of the core particle and made of a material having a refractive index lower than a refractive index of the core particle in a visible light wavelength range of 360 nm to 820 nm; a modifying layer formed on the total-reflection inducing layer; and an analyte-sensing substance bound to the modifying layer and selectively conjugated to the target analyte.

In one embodiment of the present disclosure, the core particle may have a spherical shape and may be made of a transparent oxide or a transparent polymer material. For example, the core particle may be made of one selected from a group consisting of silica, glass, polystyrene, and poly(methyl methacrylate). Further, the core particle may have an average diameter of about 700 nm to 5 μm.

In one embodiment of the present disclosure, the total-reflection inducing layer covers about 30% to 70% of a surface area of the core particle.

In one embodiment of the present disclosure, the core particle may be made of a material having a refractive index of 1.4 or above, the total-reflection inducing layer may be made of a material having a refractive index of 1.2 or lower. For example, the total-reflection inducing layer may be made of at least one selected from a group consisting of aluminum (Al), copper (Cu), gold (Au), silver (Ag), and zinc (Zn).

In one embodiment of the present disclosure, the total-reflection inducing layer may have a thickness of about 10 to 100 nm.

In one embodiment of the present disclosure, the modifying layer may be made of at least one selected from a group consisting of platinum (Pt), gold (Au), and silver (Ag), and the like. Further, the modifying layer may have a thickness of about 10 to 100 nm.

In one embodiment of the present disclosure, the analyte-sensing substance may include at least one selected from a group consisting of protein, nucleic acid, ligand and receptor.

In one embodiment of the present disclosure, when the target analyte is an antigen, the analyte-sensing substance may be an antibody or an aptamer that specifically reacts with the antigen; or when the target analyte is a genetic substance, the analyte-sensing substance may be a nucleic acid material capable of complementary-binding to the genetic substance; or when the target analyte is a cell signaling substance, the analyte-sensing substance may be a chemical ligand or a cell receptor that selectively conjugates to the cell signaling substance.

In one embodiment of the present disclosure, the optical probe may further include a magnetic layer disposed between the total-reflection inducing layer and the modifying layer.

In one aspect of the present disclosure, there is provided a bio-sensor including: an analyte fixing unit configured for fixing a target analyte; an optical probe configured for selectively conjugating to the target analyte and for retro-reflecting incident light thereto; a light source unit configured for irradiating the light to the optical probe; and an optical receiving unit configured for receiving the retro-reflected light from the optical probe.

In one embodiment of the present disclosure, the optical probe may include:

a spherical transparent core particle; a total-reflection inducing layer covering a portion of a surface of the core particle and made of a material having a refractive index lower than a refractive index of the core particle; a modifying layer formed on the total-reflection inducing layer; and an analyte-sensing substance bound to the modifying layer and selectively conjugated to the target analyte.

In one embodiment of the present disclosure, the analyte fixing unit may include: a substrate; and an analyte fixing substance disposed on the substrate and selectively conjugating to the target analyte, and the light source may oriented to irradiate light in a direction inclined by about 5 to 60° with respect to a normal line to a surface of the substrate. Further, the optical receiving unit may include: a light dividing unit configured for dividing received light into light incident into the optical probe and retro-reflected light from the optical probe; an image forming unit configured for receiving the retro-reflected light from the light dividing unit and for forming an image corresponding to the retro-reflected light; and an image analyzing unit configured for analyzing the image from the image forming unit, in this case the light dividing unit may be oriented at an angle of about 5 to 60° with respect to the normal line of the surface of the substrate and in the same orientation as an orientation of the light source.

In one aspect of the present disclosure, there is provided a method for manufacturing an optical probe for a bio-sensor, the method comprising: providing a substrate; providing transparent core particles; arranging the core particles into a single layer on a surface of the substrate; sequentially performing a deposition process of a first metal and a deposition process of a second metal on the single layer, to form a stack of a total-reflection inducing layer and a modifying layer to cover a portion of a surface of each of the core particles; binding an analyte-sensing substance to the modifying layer so that the sensing substance is selectively conjugated to a target analyte; and separating the core particles, the total-reflection inducing layer, the modifying layer, and the analyte-sensing substance from the substrate, so that the separated core particles, total-reflection inducing layer, modifying layer, and analyte-sensing substance together define the optical probe.

In one embodiment of the present disclosure, each of the transparent core particles may include a silica core particle produced via Stöber method using TEOS (tetraethylorthosilicate).

In one embodiment of the present disclosure, arranging the core particles into the single layer on the surface of the substrate may include: modifying the surfaces of the core particles to be hydrophobic and arranging the core particles into a single layer on an interface between water and air; and immersing the substrate into the water and withdrawing the substrate out of the water to allow the core particles to be attached on the surface of the substrate.

In one embodiment of the present disclosure, the first metal and the second metal may be sequentially deposited on the substrate by chemical vapor deposition (CVD) or physical vapor deposition (PVD), the first metal may include at least one selected from a group consisting of aluminum (Al), copper (Cu), gold (Au), and silver (Ag), and the second metal may include at least one selected from a group consisting of platinum (Pt), gold (Au), and silver (Ag).

In one embodiment of the present disclosure, the first metal may be deposited such that the total-reflection inducing layer covers about 30 to 70% of the surface of each of the core particles.

In one embodiment of the present disclosure, when the target analyte is an antigen, the analyte-sensing substance may be an antibody protein or an aptamer that specifically reacts with the antigen, or when the target analyte is a genetic substance, the analyte-sensing substance may be a nucleic acid material capable of complementary-binding to the genetic substance, or when the target analyte is a cell signaling substance, the analyte-sensing substance may be a chemical ligand or a cell receptor that selectively conjugates to the cell signaling substance.

In one embodiment of the present disclosure, when the analyte-sensing substance is the antibody protein, binding the analyte-sensing substance to the modifying layer may include: providing a self-assembled monolayer (SAM) having a disulfide group in one terminal or molecular structure thereof, and having a succinimide group in another terminal or molecular structure thereof; binding the self-assembled monolayer (SAM) to the modifying layer; binding amine-terminated poly(amidoamine) (PAMAM) dendrimer to the self-assembled monolayer; binding a cross-linker to the PAMAM dendrimer, wherein the cross-linker has a sulfo-NHS group (N-hydroxysulfosuccinimide group) and a diazirine group; photo-crosslinking an amine group of the antibody protein and the diazirine group of the cross-linker via irradiating of ultraviolet light.

In one embodiment of the present disclosure, the method may include forming a protective film on an exposed surface of each of the core particles to prevent a non-specific conjugation of the probe with the target analyte.

### Advantageous Effects

According to the present disclosure, the optical probe has the total-reflection inducing layer covering the portion of the surface of the transparent core particle. Thus, even when using a general light source as a non-spectroscopic source such as white mixed light, a very strong retro-reflected signal may be generated. Further, the analyte-sensing substance is formed only on the modifying layer on the surface of the optical probe. Thus, the target analyte is conjugated to the optical probe, the exposed surface of the core particle is oriented to face the light source, thereby to generate a stronger retro-reflected signal. In addition, when the magnetic layer is formed between the total-reflection inducing layer and the modifying layer, it is possible not only to control an orientation of the optical probe by applying a magnetic field from the outside, but also only to easily remove only the optical probe from the mixture by using the external magnetic field.

### BRIEF DESCRIPTION OF THE DRAWINGS

FIG. 1 is a schematic diagram illustrating a bio-sensor according to an embodiment of the present disclosure.

FIG. 2a and FIG. 2b are cross-sectional views illustrating embodiments of an optical probe shown in FIG. 1.

FIG. 3 is a flow chart illustrating a method for manufacturing an optical probe for a bio-sensor according to an embodiment of the present disclosure.

FIG. 4 is a view for illustrating an embodiment of a method for fabricating a transparent oxide core particle.

FIG. 5 is a view for illustrating an embodiment of a method for arranging the core particles in a single layer on a surface of a substrate.

FIG. 6 is a view for illustrating an embodiment of a method for binding an analyte-sensing substance, a nucleic acid substance, onto a modifying layer.

FIG. 7 is a view for illustrating an embodiment of a method for binding a biotin analyte-sensing substance onto a modifying layer.

FIG. 8 is a view for illustrating an embodiment of a method for binding an analyte-sensing substance, an antibody substance onto a modifying layer.

FIG. 9 is a schematic diagram for illustrating a bio-sensor manufactured according to the present disclosure for experiments.

FIG. 10 shows results of retro-reflected light analysis for the samples when the above-mentioned three kinds of diode lasers are used as light sources.

FIG. 11 shows result of retro-reflected light analysis for the samples using a white LED as a light source.

FIG. 12 shows fluorescence microscopic images of optical probes, wherein anti-mouse IgG labeled with fluoresce-materials is coupled to the optical probes, wherein an upper portion of FIG. 12 shows the image of the probe in which the anti-mouse IgG is coupled to the optical probe using the self-assembled monolayer (SAM), dendrimer and photo-crosslinking technique, wherein a lower portion of FIG. 12 shows the image of the probe in which the anti-mouse IgG is coupled to the optical probe only using the self -assembled monolayer (SAM) without using the photo-crosslinking technique.

FIG. 13a and FIG. 13b are images and graph showing experimental result for an experiment of Example 3.

### DETAILED DESCRIPTION

Hereinafter, an embodiment of the present disclosure will now be described in detail with reference to the accompanying drawings.

Since various modifications may be applied to the present disclosure and the present disclosure may have several embodiments, particular embodiments will be illustrated in the drawings and described. However, it will be understood that the description herein is not intended to limit the claims to the specific embodiments described, on the contrary, it is intended to cover alternatives, modifications, and equivalents as may be included within the spirit and scope of the present disclosure as defined by the appended claims. The same or similar reference numerals are used throughout the drawings and the description in order to refer to the same or similar constituent elements. In the accompanying drawings, the dimensions of the structure show an enlarged scale than actual for clarity of the disclosure.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element without departing from the teachings of the disclosure.

It will be understood that when an element or layer is referred to as being “bound to”, or “coupled to” another element or layer, it can be directly on, bound to, or coupled to the other element or layer, or one or more intervening elements or layers may be present. In addition, it will also be understood that when an element or layer is referred to as being “between” two elements or layers, it can be the only element or layer between the two elements or layers, or one or more intervening elements or layers may also be present.

Spatially relative terms, such as “beneath,” “below,” “lower,” “under,” “above,” “upper,” and the like, may be used herein for ease of explanation to describe one element or feature's relationship to another element s or feature s as illustrated in the figures. It will be understood that the spatially relative terms are intended to encompass different orientations of the device in use or in operation, in addition to the orientation depicted in the figures. For example, if the device in the figures is turned over, elements described as “below” or “beneath” or “under” other elements or features would then be oriented “above” the other elements or features. Thus, the example terms “below” and “under” can encompass both an orientation of above and below. The device may be otherwise oriented for example, rotated 90 degrees or at other orientations, and the spatially relative descriptors used herein should be interpreted accordingly.

The terminology used herein is for the purpose of describing particular embodiments only and is not intended to be limiting of the present invention. As used herein, the singular forms “a,” “an,” and “the” are intended to include the plural forms as well, unless the context clearly indicates otherwise. It will be further understood that the terms “comprises,” “comprising,” “includes,” and “including,” when used in this specification, specify the presence of the stated features, integers, steps, operations, elements, and/or components, but do not preclude the presence or addition of one or more other features, integers, steps, operations, elements, components, and/or groups thereof.

Unless otherwise defined, all terms including technical and scientific terms used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

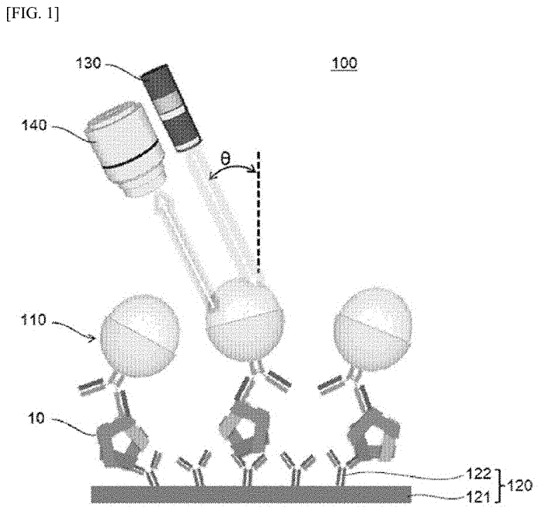

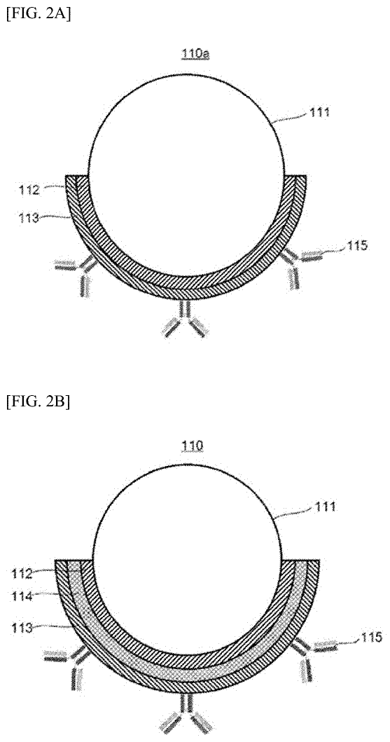

FIG. 1 is a schematic diagram illustrating a bio-sensor according to an embodiment of the present disclosure, FIG. 2a and FIG. 2b are cross-sectional views illustrating embodiments of an optical probe shown in FIG. 1.

First, referring to FIG. 1 and FIG. 2a, the bio-sensor 100 according to an embodiment of the present disclosure may detect a presence, a concentration, and the like of a target analyte 10 in an optical manner. The target analyte 10 may be not particularly limited. The analyte may include an analyte such as microorganism such as a bacterium and a virus or one or more bio-substances such as red blood cell, cell, and genetic substance containing at least one selected from a group consisting of protein, polysaccharide and lipid, etc.

The bio-sensor 100 may include an optical probe 110, an analyte fixing unit 120, a light source unit 130, and an optical receiving unit 140. In one embodiment, in the bio-sensor 100, the target analyte 10 may be fixed to the analyte fixing unit 120 and then the optical probe 110 may be selectively conjugated to the target analyte 10. Then, light generated from the light source unit 130 may be irradiated to the optical probe 110 conjugated to the target analyte 10. Thereafter, the optical receiving unit 140 may receive retro-reflected light from the optical probe 110 and analyze the received light to detect a presence, a concentration, and the like of the target analyte 10. Alternatively, in another embodiment, in the bio-sensor 100, the target analyte 10 may be firstly conjugated to the optical probe 110 and then the analyte fixing unit 120 may be conjugated to the target analyte 10. Then, the light generated from the light source unit 130 may be irradiated to the optical probe 110 conjugated to the target analyte 10. Thereafter, the optical receiving unit 140 may receive the retro-reflected light from the optical probe 110 and analyze the received light to detect the presence, the concentration, and the like of the target analyte 10.

The optical probe 110 may retro-reflect the light irradiated from the light source unit 130 toward the light source unit 130 and may be selectively conjugated to the target analyte 10. In one embodiment, the optical probe 110 may include a transparent core particle 111, a total-reflection inducing layer 112 covering a portion of the core particle 111, a modifying layer 113 formed on the total-reflection inducing layer 112, and an analyte-sensing substance 115 directly or indirectly bound to the modifying layer 113.

The core particle 111 may be formed in a spherical shape. In the present disclosure, ‘spherical shape’ means not only a perfect spherical shape with radii from a center to all points on a surface being equal to each other, but also a substantially spherical shape having a difference between a maximum radius and a minimum radius being smaller than about 10%.

In one embodiment, the core particle 111 may have an average diameter of about 700 nm to 5 μm with taking into account a conjugation property with the target analyte 10, a relation between the diameter and a wavelength of the light irradiated from the light source, and the like.

In one embodiment, the core particle 111 may be made of a transparent material capable of transmitting the incident light therethrough. For example, the core particle 111 may be made of a transparent oxide or a transparent polymer material. The transparent oxide may include, for example, silica and glass, and the like. The transparent polymer material may include, for example, polystyrene, poly methyl methacrylate, and the like.

The total-reflection inducing layer 112 is configured to cover the portion of the surface of the core particle 111. In addition, the total-reflection inducing layer 112 may increase an amount of retro-reflected light toward the light source by totally reflecting at least a portion of the light traveling into the core particle 111.

In one embodiment, the total-reflection inducing layer 112 may be formed on the surface of the core particle 111 to cover about 30% to 70% of the surface of the core particle 111. When the total-reflection inducing layer 112 covers less 30% of the total surface of the core particle 111, the amount ratio of light as is not retro-reflected and leaks into the particle, among the total light beams incident into the core particle 111, is increased. As a result, the sensitivity of the bio-sensor 100 deteriorates. Further, when the total-reflection inducing layer 112 covers more than 70% of the surface of the core particle 111, the amount of light incident into the core particle 111 decreases. As a result, the sensitivity of the bio-sensor 100 deteriorates. For example, the total-reflection inducing layer 112 may be formed on the surface of the core particle 111 so as to cover about 40% to 60% of the surface of the core particle 111.

In one embodiment, the total-reflection inducing layer 112 may be made of a material having a lower refractive index than the core particle 111 in order to increase the amount ratio of light as is retro-reflected toward the light source unit 130 by totally reflecting at least a portion of the light traveling into the core particle 111. In one embodiment, the core particle 111 may be made of a material having a refractive index of about 1.4 or higher in a visible light wavelength region of at least 360 nm to 820 nm. The total-reflection inducing layer 112 may be made of a material having a refractive index lower than that of the core particle 111.

In detail, when the core particle 111 is made of a transparent oxide or a transparent polymer material having a refractive index of about 1.4 or higher in the visible light region, the total-reflection inducing layer 112 may be made of a metal material having a lower refractive index than that of the core particle 111. For example, the total-reflection inducing layer 112 may be made of one or more metals selected from a group consisting of gold (Au) having a refractive index of about 0.22, silver (Ag) having a refractive index of about 0.15, aluminum (Al) having a refractive index of about 1.0, Copper (Cu) having a refractive index of 0.4, zinc (Zn) having a refractive index of about 1.2, and the like.

The total-reflection inducing layer 112 is preferably made of a material having a strong adhesion to the core particle 111. For example, when the core particle 111 is made of the transparent oxide, the total-reflection inducing layer 112 may be made of the aluminum (Al) or the copper (Cu).

In one embodiment, the total-reflection inducing layer 112 may have a thickness of about 10 to 100 nm in order to prevent the light leakage due to light transmission and to improve dispersibility of the optical probe 110. When the thickness of the total-reflection inducing layer 112 is less than 10 nm, a portion of the light incident into the core particle 111 may penetrate through the total-reflection inducing layer 112 and thus leak. On the other hand, when the thickness of the total-reflection inducing layer 112 is above 100 nm, a weight of the optical probe 110 may increase, thereby deteriorating the dispersibility of the optical probe 110 in a liquid.

The modifying layer 113 may be formed on a surface of the total-reflection inducing layer 112. The modifying layer 113 may be made of a metal material that is easily bonded to the analyte. For example, the modifying layer 113 may be made of a noble metal such as platinum (Pt), gold (Au), or silver (Ag), which is easily modified by the analyte and has an excellent oxidation stability.

In one embodiment, the modifying layer 113 may be formed as a separate layer independent of the total-reflection inducing layer 112. For example, when the total-reflection inducing layer 112 is made of a metal material other than a noble metal, the modifying layer 113 may be a noble metal material layer covering the total-reflection inducing layer 112. Alternatively, in another embodiment, the modifying layer 113 and the total-reflection inducing layer 112 may be integrally formed. For example, when the total-reflection inducing layer 112 is made of the noble metal having a refractive index lower than that of the core particle such as the gold (Au) or the silver (Ag), the total-reflection inducing layer 112 may also be function as the modifying layer 113.

In one embodiment, the modifying layer 113 may have a thickness of about 10 to 100 nm to prevent dispersion and aggregation of the optical probe 110 in the liquid.

The analyte-sensing substance 115 may be made of a substance that is directly or indirectly bound to the modifying layer 113 and is selectively conjugated to the target analyte 10. The analyte-sensing substance 115 may vary depending on a type of a target analyte 10 to be detected. The substance 115 may include one or more selected from a group consisting of protein, nucleic acid, ligand, and the like. In one example, when the target analyte 10 is an antigen, the analyte-sensing substance 115 may be an antibody or an aptamer that specifically reacts with the antigen. In another example, when the target analyte 10 is a genetic substance, the analyte-sensing substance 115 may be a nucleic acid material such as DNA (deoxyribonucleic acid), RNA (ribonucleic acid), and PNA (peptide nucleic acid) capable of complementary-binding to the genetic substance. In still another example, when the target analyte 10 is a cell signaling substance, the analyte-sensing substance 115 may be a chemical ligand that selectively conjugates to the cell signaling substance.

In one embodiment, the analyte-sensing substance 115 may be directly bound to the modifying layer 113. For example, when the analyte-sensing substance 115 has a functional group capable of binding to the metal of the modifying layer 113, the analyte-sensing substance 115 may be directly bound to the modifying layer 113 via the binding between the functional group and the metal of the modifying layer 113. For example, when the analyte-sensing substance 115 includes a thiol group (—SH), the analyte-sensing substance 115 may be directly bound to the modifying layer 113 via a binding between the thiol group and the metal of the modifying layer 113.

In another embodiment, the analyte-sensing substance 115 may be bound to the modifying layer 113 via an intermediate reactant such as a self-assembled monolayer having, in a molecular structure thereof, a first functional group capable of binding to the metal of the modifying layer 113 and a second functional group capable of binding to the analyte-sensing substance 115. In one example, when the analyte-sensing substance 115 includes an amine group, the intermediate reactant has, in one terminal or molecular structure thereof, a thiol group or a disulfide group which is capable of binding to the metal of the modifying layer 113, and, has, in another terminal or molecular structure thereof, a carboxyl group, a succinimide group, an aldehyde group, or the like, which is capable of binding with an amine group of the analyte-sensing substance 115.

In one embodiment, the analyte-sensing substance 115 may be coupled only onto the surface of the modifying layer 113, but not onto the exposed surface of the core particle 111. When the analyte-sensing substance 115 is bound only to the above specific position of the core particle 111 as covered by the total-reflection inducing layer 112 and the modifying layer 113, the optical probe 110 may be oriented such that the exposed portion of the core particle 111 of the optical probe 110 that is not conjugated with the target analyte 10 faces the light source unit 130, as described below. Therefore, this may induce a stronger retro-reflected signal, and as a result, the sensitivity of the bio-sensor 100 may be remarkably improved.

In accordance with the present disclosure, the analyte-sensing substance 115 may be coupled only onto the modifying layer 113 using various methods depending on a kind of the analyte-sensing substance 115. This will be described later.

In one embodiment, the optical probe 110 may further include a magnetic layer 114 disposed between the total-reflection inducing layer 112 and the modifying layer 113 as shown in FIG. 2b. The layer 114 may be made of a magnetic material. For example, the magnetic layer 114 may be made of a magnetic material such as iron (Fe), nickel (Ni), manganese (Mn), sintered bodies thereof, or oxide.

When the optical probe 110 further includes the magnetic layer 114, an orientation of the optical probe 110 may be adjusted by applying a magnetic field from the outside. Moreover, applying the magnetic field from the outside results in easy separation of only the optical probe 110 from a mixture containing the optical probe 110.

The analyte fixing unit 120 may include a substrate 121, and an analyte fixing substance 122 disposed on the substrate 121 and selectively conjugating with the target analyte 10.

A material, shape and the like of the substrate 121 are not particularly limited as long as the analyte fixing substance 122 may be coupled thereto. For example, the substrate 121 may include a silicon substrate, a glass substrate, a polymer substrate, a paper substrate, a metal substrate, and the like with a gold (Au) formed thereon. Alternatively, the substrate may include a glass substrate, a polymer substrate, a paper substrate, a metal substrate, and the like, whose surface is modified so that the analyte fixing substance 122 may be coupled to the modified surface.

The analyte fixing substance 122 may include a substance selectively conjugated to the target analyte 10. The analyte fixing substance 122 may vary depending on a type of the target analyte 10 to be detected. The substance 122 may include one or more selected from a group consisting of protein, nucleic acid, ligand, and the like. For example, when the target analyte 10 is the antigen material, the analyte fixing substance 122 may be an antibody or an aptamer material that specifically reacts with the antigen material. In another example, when the target analyte 10 is a genetic substance, the analyte fixing substance 122 may be a nucleic acid material such as DNA (deoxyribonucleic acid), RNA (ribonucleic acid), PNA (peptide nucleic acid), and the like capable of conjugating to the genetic substance in a complementary manner. In further example, when the target analyte 10 is a cell signaling substance, the analyte fixing substance 122 may be a chemical ligand that selectively conjugates to the cell signaling substance. That is, the analyte fixing substance 122 may be the same substance as the analyte-sensing substance 115, or may be another substance selectively conjugating to the target analyte 10.

In one embodiment, the analyte fixing substance 122 may be directly coupled to the substrate 121 or may be coupled to the substrate 121 via an intermediate reactant such as a self-assembled monolayer. For example, the analyte-fixing substance 122 may be coupled to the substrate 121 in the same or similar manner as the analyte-sensing substance 115 is coupled to the modifying layer 113.

Further, the analyte fixing unit 120 may further include the substrate 121 and a sidewall (not shown) extending from the substrate upwardly. The substrate 121 and the sidewall together define a space, in which a solution containing the target analyte 10 is received, and the space has an open top. The sidewall may be disposed on the substrate 121 to surround the analyte fixing substance 122 fixed to the substrate 121. A structure, shape, material and the like of the sidewall are not particularly limited as long as the space defined by the sidewall and substrate receives a solution containing the target analyte 10.

The light source unit 130 may be disposed above the analyte fixing unit 120 and may be include a light source irradiating light to the optical probe 110 conjugated with the target analyte 10 in the receiving space of the analyte fixing unit 120. As the light source, a light source that generates a mixed light mixture of various wavelengths may be used, or a light source that generates a monochromatic light of a specific wavelength may be used without limitation.

The optical receiving unit 140 is disposed above the analyte fixing unit 120 to be spaced apart from the light source unit 130. The optical receiving unit 140 may receive the light retro-reflected from the optical probe 110 among the light generated from the light source unit 130 and irradiated to the optical probe 110. Then, the optical receiving unit 140 may analyze information about the presence, concentration, and the like of the target analyte 10. A configuration of the optical receiving unit 140 is not particularly limited as long as the unit 140 receives the retro-reflected light, and analyzes the information about the target analyte 10. For example, in one embodiment, the optical receiving unit 140 may include a microscope that may directly identify the retro-reflected light. Alternatively, in another embodiment, the optical receiving unit 140 may include an image forming unit for imaging the retro-reflected optical signal and an image analyzing unit analyzing image generated by the image forming unit. In still another embodiment, the optical receiving unit 140 may include a light dividing unit for dividing received light into the incident light incident into the optical probe 110 from the light source unit 130 and the retro-reflected light from the optical probe 110, a lens capable of focusing and enlarging the optical signal divided from the light dividing unit, an image forming unit for receiving and imaging the enlarged optical signal and an image analyzing unit for analyzing image generated by the image forming unit.

In one embodiment, in order to remove an effect of light being mirror-reflected from the substrate 121 of the analyte fixing unit 120, the light source unit 130 may irradiate the light in a direction inclined by about 5 to 60° with respect to a normal line to the surface of the substrate 121 onto which the analyte fixing substance 122 is bound.

In this connection, when the optical receiving unit 140 includes the light dividing unit, the image forming unit, and the image analyzing unit, the light source unit 130 may be oriented to irradiate the light in a direction inclined by about 5 to 60° with respect to a normal line to the surface of the substrate 121. The light dividing unit may also be oriented to be inclined at a predetermined angle with respect to the normal line to the surface of the substrate 121 to minimize an effect of a mirror reflection of light from the light dividing unit.

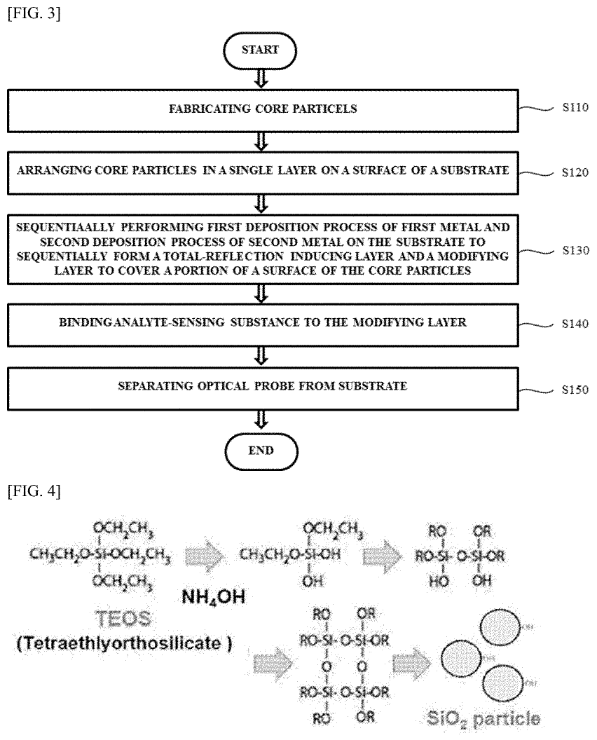

FIG. 3 is a flow chart illustrating a method for manufacturing an optical probe for a bio-sensor according to an embodiment of the present disclosure. The optical probe manufactured by the above manufacturing method has the same structure as the optical probe 110 shown in FIG. 1, FIG. 2a and FIG. 2b.

Referring to FIG. 3 together with FIG. 1, FIG. 2a and FIG. 2b, the method for manufacturing the optical probe 110 for the bio-sensor according to an embodiment of the present disclosure includes: a step S110 for fabricating the core particles 111; a step S120 for arranging the core particles 111 in a single layer on the surface of the substrate; a step S130 for sequentially performing a deposition process of a first metal and a deposition process of a second metal on the substrate on which the core particles 111 are arranged, to form a stack of the total-reflection inducing layer 112 and the modifying layer 113 to cover the portion of the surface of the core particles 111, wherein the first and second metals correspond to the inducing layer 112 and modifying layer 113 respectively; a step S140 for binding the analyte-sensing substance 115 to the modifying layer 113; and a step S150 for separating from the substrate the optical probe 110 together with the analyte-sensing substance 115 coupled thereto.

In the step S110 of fabricating the core particles 111, the core particles 111 may be synthesized by a chemical method.

In an embodiment, when the core particles 111 are made of the transparent oxide, the core particles may be fabricated using a Stöber method or a seed-growth method. FIG. 4 is a view for illustrating an embodiment of a method for fabricating a transparent oxide core particle, which illustrates a method for fabricating the silica core particles by the Stöber method using TEOS (tetraethylorthosilicate).

In another embodiment, when the core particles 111 are made of the transparent polymer material, the core particles 111 may be produced by suspension polymerization, dispersion polymerization, emulsion polymerization, precipitation polymerization, or the like.

The core particles 111 fabricated by the above method may have a spherical shape having an average diameter of about 700 nm to 5 μm.

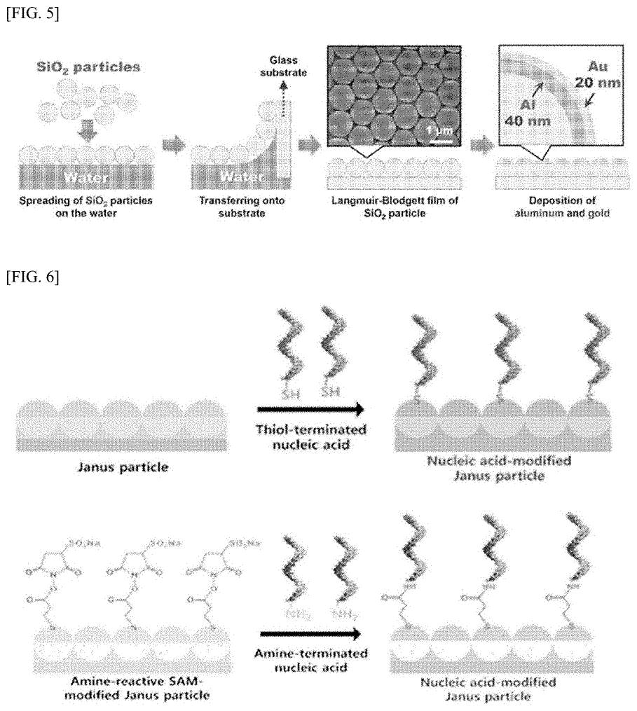

In the step S120 for arranging the core particles 111 in the single layer on the surface of the substrate, the core particles 111 may be arranged in a single layer on the substrate using a Langmuir-Blodgett film process as shown in FIG. 5. For example, arranging the core particles 111 in the single layer on the surface of the substrate may be executed as follows: the surface of the core particles 111 may be modified to have a hydrophobic property, and then the modified core particles may be densely arranged in the single layer on an interface between water and air so that a Langmuir-Blodgett film of the core particles 111 may be formed on the interface and transferred to the substrate.

In the step S130 for forming the total-reflection inducing layer 112 and the modifying layer 113, first, the deposition process of the first metal on the substrate on which the core particles 111 are arranged in the single layer may be performed to form the total-reflection inducing layer 112. Then, the deposition process of the second metal may be performed on the total-reflection inducing layer 112, to form the modifying layer 113.

The first metal and the second metal may be deposited on the substrate using chemical vapor deposition (CVD) or physical vapor deposition (PVD) methods. However, when the core particles 111 have a low thermal stability, the first metal and the second metal are preferably deposited using the physical vapor deposition (PVD), which may be performed at a relatively lower temperature than the chemical vapor deposition (CVD). Alternatively, the first metal and the second metal may be deposited on the substrate by thermal evaporation deposition, sputtering deposition, e-beam physical vapor deposition (EBPVD) or the like. The first metal may be a metal having a refractive index of about 1.4 or above such as aluminum (Al), copper (Cu), gold (Au), silver (Ag) or the like. The second metal may be a noble metal such as platinum (Pt), gold (Au), silver (Ag) or the like.

In order to prevent the light transmission through the total-reflection inducing layer 112 and to improve the dispersibility of the optical probe 110 in the liquid, the first metal may be deposited to a thickness of about 10 to 100 nm. The second metal may be deposited to a thickness of about 10 to 100 nm to prevent the dispersion and the aggregation of the optical probe 110 in the liquid.

When the spherical core particles 111 are arranged to form the Langmuir-Blodgett film on the substrate as described above and then the first and second metals are deposited thereon, the total-reflection inducing layer 112 and the modifying layer 113 may be formed to cover about 30 to 70% of the surface of the core particles 111 by adjusting the deposition thickness of the first and second metals.

In the step S140 for binding the analyte-sensing substance 115 to the modifying layer 113, the analyte-sensing substance 115 may be a substance capable of selectively binding to the target analyte 10. For example, when the target analyte 10 is the antigen material, the analyte-sensing substance 115 may be the antibody or the aptamer that specifically reacts with the antigen. In another example, when the target analyte 10 is the genetic substance, the analyte-sensing substance 115 may be the nucleic acid material such as the DNA (deoxyribonucleic acid), RNA (ribonucleic acid), and PNA (peptide nucleic acid) capable of complementary binding to the genetic substance. In further example, when the target analyte 10 is the cell signaling substance, the analyte-sensing substance 115 may be the chemical ligand that selectively conjugates to the cell signaling substance.

The analyte-sensing substance 115 may be directly or indirectly bound to the modifying layer 113. Specifically, when the analyte-sensing substance 115 has the functional group capable of binding to the metal of the modifying layer 113, the analyte-sensing substance 115 may be directly bound to the modifying layer 113 via the binding between the functional group and the metal of the modifying layer 113. In another example, the analyte-sensing substance 115 may be bound to the modifying layer 113 via the intermediate reactant such as the self-assembled monolayer having, in a molecular structure thereof, the first functional group capable of binding to the metal of the modifying layer 113 and the second functional group capable of binding to the analyte-sensing substance 115.

The analyte-sensing substance 115 may be coupled only onto the surface of the modifying layer 113, but not onto the exposed surface of the core particle 111.

In one embodiment, when the analyte-sensing substance 115 is the nucleic acid material such as the DNA, RNA, PNA, and the like that selectively reacts with the genetic substance, the analyte-sensing substance 115 may be coupled to the modifying layer 113 as shown in FIG. 6 as follows: (i) the thiol group (—SH) may be coupled to the nucleic acid material, and, then, the nucleic acid material may be bound to the metal of the modifying layer 113 via the introduced thiol group; alternatively, (ii) the self-assembled monolayer is prepared, in which the monolayer has, in one terminal or molecular structure thereof, the thiol group or the disulfide group and has, in opposite another terminal or molecular structure thereof, at least one functional group selected from a group consisting of the carboxyl group, the succinimide group, the aldehyde group and the like; then, the self-assembled monolayer is coupled, on one side, to the modifying layer 113 via the thiol group or the disulfide group thereof, while the self-assembled monolayer is coupled, on the other side, to the nucleic acid material having the amine group via the at least one functional group selected from a group consisting of the carboxyl group, the succinimide group, the aldehyde group and the like thereof.

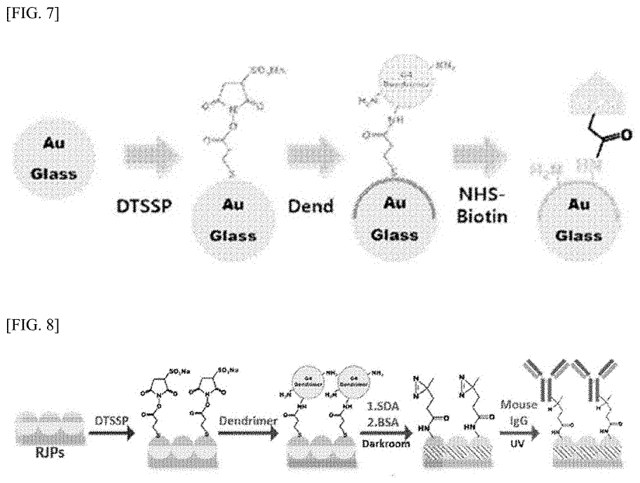

In other embodiment, when the analyte-sensing substance 115 is the chemical ligand that selectively reacts with the cell signaling substance, the analyte-sensing substance 115 may be coupled to the modifying layer 113 as shown in FIG. 7 as follows: the self-assembled monolayer (SAM) is prepared which has the disulfide group in one terminal or molecular structure thereof and having the succinimide group or the aldehyde group in another terminal or molecular structure thereof, and, then, the SAM is bound only to the surface of the modifying layer 113 via the disulfide group thereof, and, amine-terminated PAMAM dendrimer (amine-terminated poly(amidoamine) dendrimer) is bound to the self-assembled monolayer, and then the chemical ligand having the succinimide group is bound to the amine-terminated PAMAM dendrimer. FIG. 7 shows an embodiment of a method of selectively binding biotin only to the modifying layer 113 in the same manner as described above.

In another embodiment, when the analyte-sensing substance 115 is the antibody protein that selectively conjugates to the antigen, a protein such as the antibody requires a more sophisticated conjugating method because the antibody protein is non-specifically bounded to the core particle 111 and the modifying layer 113 while the nucleic acid or the chemical ligand is specifically bounded to the core particle 111 and the modifying layer 113. In this case, the analyte-sensing substance 115 may be coupled to the modifying layer 113 as shown in FIG. 8 as follows: specifically, the self-assembled monolayer (SAM) is prepared which has the disulfide group in one terminal or molecular structure thereof and has the succinimide group in another terminal or molecular structure thereof; then, the SAM may be bound only to the surface of the modifying layer 113 via a binding between the disulfide group of the self-assembled monolayer (SAM) and the metal of the modifying layer 113; then, the amine-terminated PAMAM dendrimer (amine-terminated poly (amidoamine) dendrimer) is bound to the SAM; then, a cross-linker having a sulfo-NHS group (N-hydroxysulfosuccinimide group) and a diazirine group is bound to the PAMAM dendrimer via covalent bonding between the amine group of the PAMAM dendrimer and the sulfo-NHS group of the cross-linker; and, then, BSA-containing solution is treated in a dark condition such that the exposed surface of the core particle 111 on which the modifying layer 113 is not formed is protected; thereafter, the antibody protein into which the amine group has been introduced is added into the BSA-containing solution, and, then, ultraviolet light is irradiated to the BSA-containing solution, to induce a photo-crosslinking between the diazirine group of the cross-linker and the amine group of the antibody protein, thereby binding the antibody protein to the cross-linker. In this connection, the cross-linker may include sulfo-NHS-diazirine (SDA), NHS-SS-Diazirine (SDAD), sulfo-NHS-SS-Diazirine (sulfo-SDAD), N-5-azido-2-nitrobenzoyloxysuccinimide (ANB-NOS), sulfosuccinimidyl 6-(4′-azido-T-nitrophenylamino)hexanoate (sulfo-SANPAH), and the like. Further, in order to block unreacted cross-linker residues, ethanolamine may be added into the solution, and the ultraviolet light may be irradiated again.

In the step S150 for separating the optical probe 110 from the substrate, the optical probe 110 manufactured as described above may be separated from the substrate by an ultrasonic treatment.

In this connection, in the optical probe 110 separated from the substrate, a protective layer may be formed on the exposed surface of the core particle 111 and the surface of the modifying layer 113 to prevent a non-specific conjugation with the target analyte. For example, by immersing the optical probe 110 into phosphate buffer solution containing bovine serum albumin (BSA), the protective film may be formed on the exposed surface of the core particle 111 and the surface of the modifying layer 113.

Hereinafter, examples of the present disclosure will be described in detail. However, the following examples are for some embodiments of the present disclosure, and the present disclosure is not to be construed as being limited to the following examples.

### EXAMPLE 1

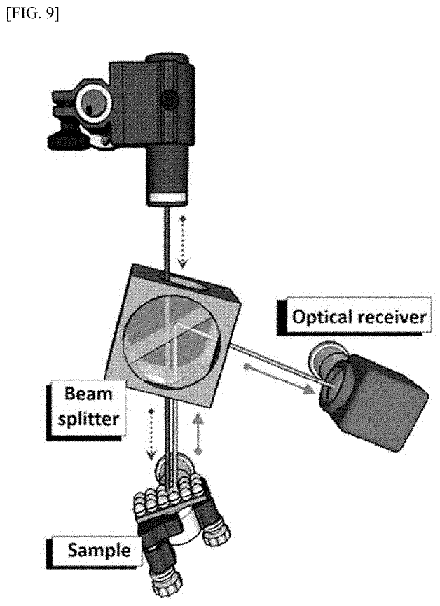

As samples, a carbon tape with first particles containing the spherical silica core particles and an aluminum total-reflection inducing layer coating 50% of surface of the spherical silica core particles, a carbon tape with second particles consisting of only the silica core particles and a carbon tape with no particles attached were prepared. Then, in order to analyze these samples, a bio-sensor as shown in FIG. 9 was manufactured. In this connection, the silica core particle coated with the aluminum total-reflection inducing layer was attached to the carbon tape such that an exposed surface of the core particle faced the light source.

In the bio-sensor, the light source, a beam splitter and the sample were arranged in a line. In order to eliminate the influence of the mirror reflection occurring on surfaces of the samples, the samples were installed at an angle of 30° to the right with respect to the traveling direction of the incident light. The beam splitter is also installed at an angle of 25° to the right with respect to the traveling direction of the incident light in order to exclude the influence of various mirror reflections that may occur from the beam splitter. In addition, a portable spectrometer was used as the optical receiving unit in order to analyze an amount of the retro-reflected light.

The amounts of the retro-reflected light were measured for each of the above samples using a diode laser having a wavelength of 405 nm, a diode laser having a wavelength of 532 nm, a diode laser having a wavelength of 655 nm, and a white LED as the light source.

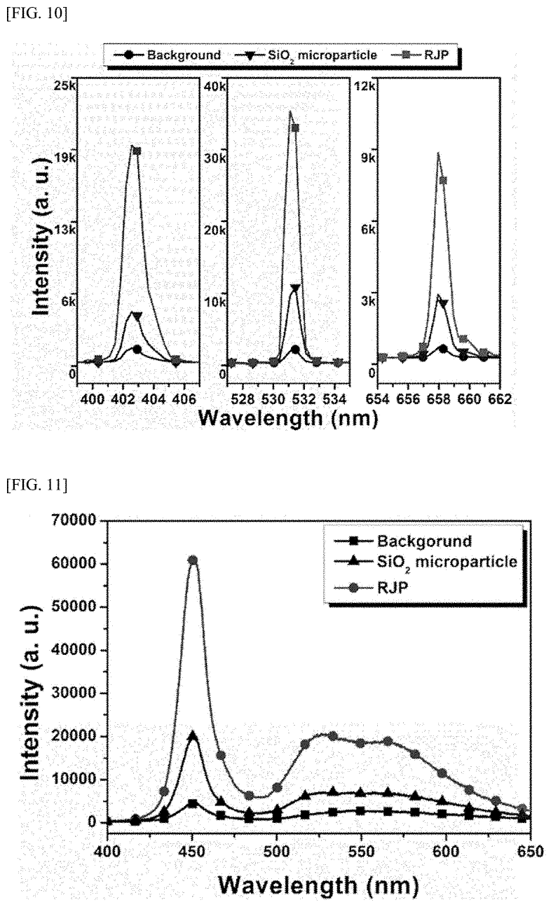

FIG. 10 shows results of the retro-reflected light analysis for the above samples when using the above three types of the diode lasers as the light source. FIG. 11 shows result the retro-reflected light analysis for the above samples when using the white LED as the light source.

Referring to FIG. 10, the strongest retro-reflected signal was detected on the carbon tape with the silica core particle coated with the aluminum total-reflection inducing layer and the retro-reflected signal was not detected on the carbon tape without the particles. Specifically, as a result of quantitative analysis, the carbon tape with the silica core particles coated with the aluminum total-reflection inducing layer at each of wavelengths of 405 nm, 532 nm and 655 nm provided 458%, 246% and 180% stronger retro-reflected signals than the carbon tape with the pure silica particles, respectively.

Referring to FIG. 11, retro-reflected signal analysis for the white LED light source was similar to that of the diode laser light sources, the carbon tape with the silica core particles coated with the aluminum total-reflection inducing layer provided stronger retro-reflected signal than the carbon tape with the pure silica particles at all wavelengths.

To sum up the above, it was confirmed that when the total-reflection inducing layer is covered on the core particle, the amount of the retro-reflected light may increase significantly and the above-mentioned particles may be used as optical probes in all visible light regions.

### EXAMPLE 2

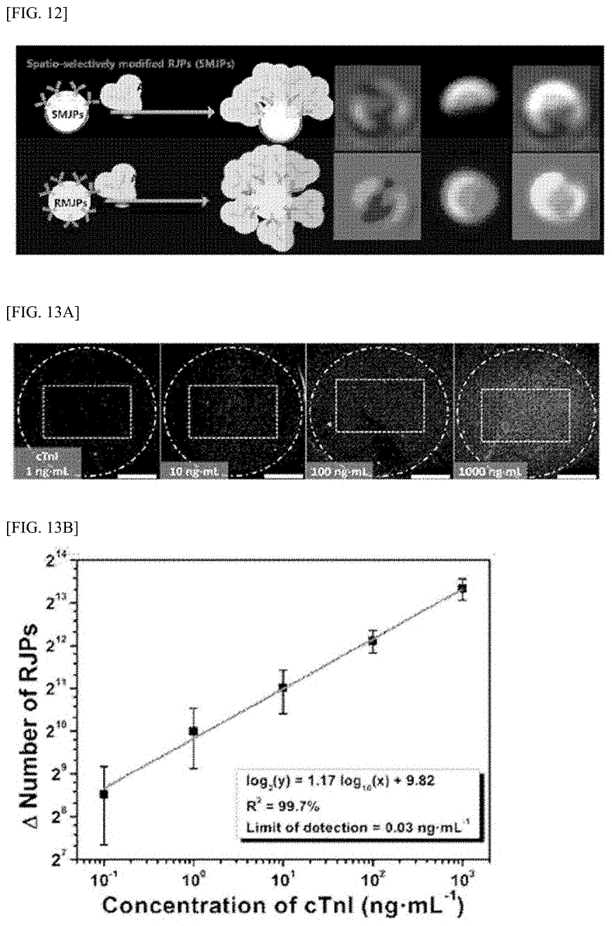

FIG. 12 shows fluorescence microscopic images of optical probes, wherein anti-mouse IgG labeled with fluoresce-materials is coupled to the optical probes, wherein an upper portion of FIG. 12 shows the image of the probe in which the anti-mouse IgG is coupled to the optical probe using the self-assembled monolayer (SAM), dendrimer and photo-crosslinking technique, wherein a lower portion of FIG. 12 shows the image of the probe in which the anti-mouse IgG is coupled to the optical probe only using the self-assembled monolayer (SAM) without using the photo-crosslinking technique.

Referring to FIG. 12, it was confirmed that the antibody protein (mouse IgG) was bound only onto the modifying layer in the optical probe in which the anti-mouse IgG is coupled to the optical probe using the self-assembled monolayer (SAM), dendrimer and photo-crosslinking technique. However, it was confirmed that the antibody protein (mouse IgG) was bound not only onto the modifying layer but also onto the exposed surface of the silica core particle in the optical probe in which the anti-mouse IgG is coupled to the optical probe only using the self-assembled monolayer (SAM) without using the photo-crosslinking technique. Thus, when the analyte-sensing substance is the antibody protein that selectively conjugates to the antigen, the photo-crosslinking technique may result in the position-specific binding of the antibody protein only to the modifying layer.

### EXAMPLE 3

A sandwich immunoassay for cTnI as a myocardial infarction biomarker, was performed. Specifically, a cTnI-immobilizing antibody is bound to a surface of a gold chip having the amine-reactive self-assembled monolayer (SAM) formed thereon. The gold chip reacted with various concentrations of cTnI 0 to 1000 ng/mL which are bound to the cTnI-immobilizing antibody. Then, an optical probe having an antibody for detecting the cTnI was conjugated to the cTnI of the gold chip. Thereafter, an image on the corresponding sensing substrate was obtained using an ×5 object lens and a white LED light source on an optical microscope.

FIG. 13a and FIG. 13b are images and graph showing experimental result for the experiment.

Referring to FIGS. 13a and 13b, as a concentration of the cTnI increases, the number of the optical probes present on the sensing substrate increased. The result of counting the optical probes using an Image J program is shown in FIG. 13b. FIG. 13b shows a result of averaging the results of the experiments repeated three times. A detection sensitivity of produced retro-reflection-based cTnI immunosensing system was calculated to be 0.03 ng/mL, which meets cut-off level, 0.1 ng/mL in a myocardial infarction. In contrast to conventional cTnI optical bio-sensors that detect the cTnI using a complex spectroscopic analysis system, immunosensing techniques implemented in the present disclosure use only the minimal optical system and non-spectral white light sources, detection of clinically meaningful biomarkers could be implemented.

Although the present disclosure has been described with reference to preferred embodiments, workers skilled in the art will recognize that changes may be made in form and detail without departing from the spirit and scope of the present disclosure.

1.-13. (canceled)

14. A bio-sensor comprising:
an analyte fixing unit configured to fix a target analyte;
an optical probe configured to selectively conjugate to the target analyte and to retro-reflect incident light thereto;
a light source unit configured to irradiate light to the optical probe; and
an optical receiving unit configured to receive the retro-reflected light from the optical probe.

15. The bio-sensor of claim 14, wherein the optical probe comprises:
a transparent core particle;
a total-reflection inducing layer covering a portion of a surface of the core particle, wherein the inducing layer is made of a material having a refractive index lower than a refractive index of the core particle with respect to a visible light wavelength range of 360 nm to 820 nm;
a modifying layer on the total-reflection inducing layer; and
an analyte-sensing substance bound to the modifying layer, wherein the analyte-sensing substance is selectively conjugated to the target analyte.

16. The bio-sensor of claim 14, wherein the analyte fixing unit comprises a substrate; and an analyte fixing substance disposed on the substrate and configured to be selectively conjugated to the target analyte,
wherein the light source unit irradiates the light in a direction inclined by about 5° to 60° with respect to a normal line to a surface of the substrate.

17. The bio-sensor of claim 16, wherein the optical receiving unit comprises a light dividing unit configured to divide received light into first light incident into the optical probe and second light retro-reflected from the optical probe;
an image forming unit configured to receive the second light from the light dividing unit and to form an image corresponding to the second light; and
an image analyzing unit configured to analyze the image formed by the image forming unit,
wherein the light dividing unit is oriented at an angle of about 0° to 60° with respect to the normal line of the surface of the substrate, which is orientated in a same or adjacent direction of the light irradiated by the light source.

18.-25. (canceled)
